(19) United States
(12) Patent Application Publication
(10) Pub. No.: US 2004/0091470 A1
(43) Pub. Date: May 13, 2004

(54) Use of bacterial phage associated lytic enzymes to prevent food poisoning

(76) Inventors: Vincent Fischetti, West Hempstead, NY (US); Lawrence Loomis, Columbia, MD (US)

Correspondence Address: Jonathan E. Grant, 2107 Hounds Run Place, Silver Spring, MD, 20906, US

(21) Appl. No.: 10/394,574

(22) Filed: Mar. 24, 2003

Related U.S. Application Data
(63) Continuation of application No. 09/704,148, filed on Nov. 2, 2000, now abandoned.

(51) Int. Cl. A61K 38/47; A23K 1/165; A23K 1/17

(52) U.S. Cl. 424/94.61; 424/442

(57) ABSTRACT

The present invention discloses a method and composition for the treatment of bacterial contamination of food by the use of a phage associated lysing enzyme, preferably blended with an appropriate carrier. The method for treating food stuffs comprises treating the food stuffs with an anti-infection agent comprising an effective amount of at least one lytic enzyme produced by a bacteria infected with a bacteriophage specific for the bacteria. The lytic enzyme is preferably in an environment having a pH which allows for activity of said lytic enzyme. The lytic enzyme can be used for the treatment or prevention of various strains of Staphylococcus, Streptococcus, Listeria, Salmonella, E. coli, Campylobacter, Pseudomonas, Brucella, other bacteria and any combination thereof. Feed for livestock, poultry and beef in slaughterhouses, canned and bottled goods, salad bars, and eggs are just some of the food items that can be treated with at least one lytic enzyme to reduce the risk of food contamination by bacteria.

### BACKGROUND OF THE INVENTION

1. Field of the Invention

The present invention discloses a method and composition to prevent food poisoning by the use of phase associated lysing enzymes and modified versions of the lysing enzymes.

2. Description of the Prior Art

Bacterial contamination is a serious problem in the food industry. It is estimated that each year, thousands of people in the United States, and millions worldwide, die from ingesting contaminated food and drinking water. As the population of the world continues to grow, and as cities become more crowded and agricultural land becomes more scarce, there has been an increase in the amount of food that must be processed and the amount of intensive fanning which must be done, thereby resulting in the increase of food contamination. In the United States, the number of chickens infected by. Salmonella, beef infected with E. coli, and the number of rivers, streams and bays infected by farm run-off, have been rising each of the last several years.

In the past, antibiotics have been used to treat various bacterial infections. The work of Selman Waksman in the introduction and production of Streptomycetes, Dr. Fleming's discovery of penicillin, are well known as well as the work of numerous others in the field of antibiotics. Over the years, there have been additions and chemical modifications to the “basic” antibiotics in attempts to make them more powerful, or to treat people allergic to these antibiotics.

These antibiotics are have been incorporated into feedstuffs for cattle, chicken, and turkeys to prevent illnesses in the animals before they get to the slaughter houses. However, as more antibiotics have been prescribed or used at an ever increasing rate for a variety of illnesses, increasing numbers of bacteria have developed a resistance to antibiotics. Larger doses of stronger antibiotics are now being used to treat ever more resistant strains of bacteria. Multiple antibiotic resistant bacteria have consequently developed. The use of more antibiotics and the number of bacteria showing resistance has led to increasing the amount of time that the antibiotics need to be used. Broad, non-specific antibiotics, some of which have detrimental effects on the animals, are now being used more frequently.

Once these animals are slaughtered and arrive on the dinner tables of millions of people world wide, there remain chemical remnants of the antibiotics in the food. As many individuals are allergic to antibiotics, they suffer numerous medical problems when the food is ingested, such as diarrhea, headaches, stomach aches, hives, etc. Turkeys are notorious for retaining a high level of antibiotics.

The introduction of infectious agents also occurs in meat processing plants. The “fecal baths” in chicken processing plants and the bacterial contamination in beef processing plants, particularly in the production of hamburger meat, remain notorious in the food industry. Of course, bacterial contamination of food can be found along other locations of the food processing chain, such as at salad bars, where individual customers often handle the food and then place it back on the table, thereby infecting the salad with Listeria, Salmonella, E. coli, Staphylococcus, or Streptococcus. Chicken eggs are often contaminated with Salmonella. Numerous bacteria can infect the water with which food is prepared. Scientists, consumers, and grocers are finding that fish are frequently contaminated with bacteria. This problem has increased as waste from the suburbs and from agribusinesses and industrial farms washes into the Chesapeake Bay.

Additionally, other food stuffs can suffer from contamination. Salad bars are often unsanitary. Canned and bottled goods are also food stuffs which frequently become contaminated, either before or after the containers are opened by consumers.

Attempts have been made to treat bacterial diseases with by the use of bacteriophages. U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses a procedure of improved dental hygiene which comprises introducing into the mouth bacteriophages parasitic to bacteria which possess the property of readily adhering to the salivary pellicle.

It is to be noted that the direct introduction of bacteriophages into an animal to prevent or fight diseases has certain drawbacks. Specifically, the bacteria must be in the right growth phase for the phage to attach. Both the bacteria and the phage have to be in the correct and synchronized growth cycles. Additionally, there must be the right number of phages to attach to the bacteria; if there are too many or too few phages, there will either be no attachment or no production of the lysing enzyme. The phage must also be active enough. The phages are also inhibited by many things including bacterial debris from the organism it is going to attack. Further complicating the direct use of bacteriophage to treat bacterial infections is the possibility of immunological reactions, rendering the phage non-functional.

Consequently, others have explored the use of other safer and more effective means to treat and prevent bacterial infections.

U.S. Pat. No. 6,017,528 (Fischetti, et. al.), U.S. Pat. No. 5,997,862 (Fischetti et al.), and U.S. Pat. No. 5,985,271 (Fischetti et al.) disclose the use of an oral delivery mode, such as a candy, chewing gum, lozenge, troche, tablet, a powder, an aerosol, a liquid or a liquid spray, containing a lysin enzyme produced by group C streptococcal bacteria infected with a C1 bacteriophage for the prophylactic and therapeutic treatment of Streptococcal A throat infections, commonly known as strep throat.

U.S. patent application Ser. No. 09/395,636 (Fischetti et al.) discloses a method for the prophylactic and therapeutic treatment of bacteria infections which comprises the treatment of an individual with an effective amount of a lytic enzyme composition specific for the infecting bacteria, with the lytic enzyme comprising an effective amount of lytic enzyme, wherein the lytic enzyme is in an environment having a pH which allows for activity of said lytic enzyme; and a carrier for delivering said lytic enzyme. This method and composition can be used for the treatment of upper respiratory infections, skin infections, wounds, and burns, vaginal infections, eye infections, intestinal disorders and dental problems.

However, no one has used an phage associated enzyme to prevent or treat bacterial infections in the food chain.

### SUMMARY OF THE INVENTION

More specifically, the present invention discloses the use of modified versions of bacterial pahge associated lytic enzymes, which may include holin enzymes, chimeric enzymes, and shuffled enzymes to prevent bacterial infections of food, food products, livestock, chicken, or anywhere else in the food chain.

The method for obtaining and purifying the lytic enzyme produced by a bacteria infected with the bacteriophage is known in the art. Some recent evidence suggests that the phage enzyme that lyses the streptococcus organism may actually be a bacterial enzyme that is used to construct the cell wall and the phage. While replicating in the bacterium, a phage gene product may cause the upregulation or derepression of the bacterial enzyme(s) for the purpose of releasing the bacteriophage. These bacterial enzymes may be tightly regulated by the bacterial cell and are used by the bacteria for the construction and assembly of the cell wall.

The use of these lytic enzymes to prevent bacteria growth in food, however, has not been explored. Consequently, the present invention discloses the extraction and use of a variety of bacterial phage associated lytic enzymes, holin enzymes, chimeric enzymes, and shuffled enzymes for the treatment or prevention of bacterial infection of food stuffs in the food processing chain. More specifically, the present invention discloses the use of an unmodified and of modified versions of bacterial phage associated lytic enzymes, which may include unmodified lytic enzymes, holin enzymes, chimeric enzymes, and shuffled enzymes to prevent bacterial infections of food, food products, livestock, chicken, or anywhere else in the food chain.

The use of phage associated lytic enzymes produced by the infection of a bacteria with a bacteria specific phage has numerous advantages for the treatment of specific bacteria. As the phage are targeted for specific bacteria, the lytic enzymes do not interfere with normal flora. Also, lytic phages primarily attack cell wall structures which are not affected by plasmid variation. The actions of the lytic enzymes are fast and do not depend on bacterial growth.

Shuffled enzymes are enzymes where more than one sequence of usually more than one particular enzyme has been cleaved in one or more locations, and reconstructed in a specific or random order, increasing their activity.

In a preferred embodiment of the invention, shuffled enzymes are used to treat bacterial infections, thereby increasing the speed and efficiency with which the bacteria are killed.

Chimeric enzymes are enzymes which are a combination of two or more enzymes having two or more active sites such that the chimeric enzyme can act independently on the same or different molecules. This will allow for potentially treating two or more different bacterial infections at the same time. Chimeric enzymes may also be used to treat one bacterial infection by cleaving the cell wall in more than one location.

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. 1998 Jul. 1, 164(1); 159-67.

In another experiment an active chimeric cell wail lytic enzyme (TSL) has been constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumococcal cell walls.

A preferred embodiment of this invention discloses the use of chimeric lytic enzymes to treat two infectious bacteria at the same time, or to cleave the cell wall of a bacteria in two different locations.

Holin enzymes produce holes in the cell membrane. More specifically, holins form lethal membrane lesions that terminates respiration. Like the lytic enzymes, the holin enzymes are coded for and carried by a phage. In fact, it is quite common for the genetic code for the holin enzyme is found next to or even within the code for the lytic enzyme in the phage. Most holin sequences are short, and overall, hydrophobic in nature, with a highly hydrophilic carboxy-terminal domain. In many cases, the putative holin is encoded on a different reading frame within the enzymatically active domain of the phage. In other cases, the holin is encoded on the DNA next or close to the DNA coding for the phage. The holin is frequently synthesized during the late stage of phage infection and found in the cytoplasmic membrane where it causes membrane lesions.

Holins can be grouped into two general classes based on primary structure analysis. Class I holins are usually 95 residues or longer and may have three potential transmembrane domains. Class II holins are usually smaller, at approximately 65-95 residues, and the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all of the phages. Holins have been shown to be present or suggested for among others, lactococcal bacteriophage Tuc2009, lactococcal φLC3, pneumococcal bacteriophage EJ-1, Lactobacillus gasseri bacteriophage φadh, Staphylococcus aureus bacteriophage Twort, Listeria monocytogenes bacteriophages, pneumococcal phage Cp-1, Bacillus subtillis phage Φ29, Lactobacillus delbrueckki bacteriophage LL-H lysin, and bacteriophage φ11 of Staphylococcus aureus. (Loessner, et al., Journal of Bacteriology, August 1999, p. 4452-4460).

In another embodiment of the invention, holin enzymes are used in conjunction with the lytic enzymes to accelerate the speed and efficiency at which the bacteria are killed. Holin enzymes may also be in the form of chimeric and/or shuffled enzymes. Holin enzymes may also be used alone in the treatment of bacterial infections.

It should be noted that in this patent, for the sake of simplicity, holin enzymes, chimeric enzymes, and shuffled enzymes may be referred to as modified versions of the lytic enzyme.

It is an object of the invention to use phage associated lytic enzymes, holin enzymes, chimeric enzymes, shuffled enzymes, or combinations thereof to prevent bacterial infections of food.

In one embodiment of the invention, phage associated lytic enzymes holin enzymes, chimeric enzymes, shuffled enzymes, or combinations thereof are used to treat feed stuffs used to feed cattle, chickens, sheep or other live stock.

In another embodiment of the invention salad bars are treated with phage associated lytic enzymes, holin enzymes, chimeric enzymes, shuffled enzymes, or combinations thereof to prevent the growth or to kill contaminating bacteria.

In yet another embodiment of the invention, eggs are treated with a phage associated lytic enzyme, holin enzymes, chimeric enzymes, shuffled enzymes, or combinations thereof to prevent or kill. Salmonella and other bacterial contamination.

The invention also proposes spraying or incorporating a phage associated lytic enzymes, holin enzymes, chimeric enzymes, shuffled enzymes, or combinations thereof in ground beef to kill or prevent the growth of E. coli.

Another embodiment of the invention proposes spraying a phage associated lytic enzymes, holin enzymes, chimeric enzymes, shuffled enzymes, or combinations thereof over beef and chicken carcasses in slaughterhouses, or bathing the beef and chicken carcasses in a pool containing the appropriate phage associated lytic enzymes.

The phage associated lytic enzymes, holin enzymes, chimeric enzymes, shuffled enzymes, or combinations thereof can also be added to canned goods to kill or prevent the growth of certain bacteria, and to bottled goods to prevent food from turning rancid.

Additionally, phage associated lytic enzymes, holin enzymes, chimeric enzymes, shuffled enzymes, or combinations thereof can be added to bottled water to prevent the growth of bacteria.

The invention (which incorporates U.S. Pat. No. 5,604,109 in its entirety by reference) uses an enzyme produced by the bacterial organism after being infected with a particular bacteriophage to lyse specific bacteria. The present invention is based upon the discovery that lytic enzymes specific for bacteria infected with a specific phage can effectively and efficiently break down the cell wall of the bacterium in question. At the same time, the semipurified enzyme is lacking in proteolytic enzymatic activity and therefore non-destructive to mammalian proteins and tissues when present during the digestion of the bacterial cell wall.

In one embodiment of the invention the treatment of a variety of food contaminants, including Staphylococcus aureus, E. Coli, Salmonella, Listeria, Campylobacter and Brucella are disclosed. The phase associated lytic enzymes, holin enzymes, chimeric enzymes, shuffled enzymes, or combinations thereof are put in a variety of carriers and administered according to need.

In one embodiment of the invention, a feed stock comprises at least one lytic enzyme, holin enzyme, chimeric enzyme; shuffled enzyme, or combinations thereof produced by a bacteria infected with a bacteriophage specific for said bacteria.

More specifically, in one embodiment of the invention, the feed stock of cattle is treated with at least one phage associated lytic enzyme, holin enzyme, chimeric enzyme, shuffled enzyme, or combinations thereof.

In another embodiment of the invention, the feed stock of chickens is treated with at least one phage associated lytic enzyme, holin enzymes, chimeric enzymes, shuffled enzymes, or combinations thereof.

In yet another embodiment of the invention, the feed stock of turkeys is treated with at least one phage associated lytic enzyme, holin enzyme, chimeric enzyme, shuffled enzyme, or combinations thereof. Similarly, the feed stock of hogs is treated with at least one phage associated lytic enzyme, holin enzyme, chimeric enzyme, shuffled enzyme, or combinations thereof.

In another embodiment of the invention, eggs are dipped in or sprayed with a solution or liquid containing at least one phage associated lytic enzyme, holin enzyme, chimeric enzyme, shuffled enzyme, or combinations thereof.

In another embodiment of the invention, a salad bar contains salad treated with at least one lytic enzyme, holin enzyme, chimeric enzyme, shuffled enzyme, or combinations thereof

In yet another embodiment of invention, a bacteria resistant ground beef contains at least one lytic enzyme produced by a bacteria infected with a bacteriophage specific for that bacteria, holin enzyme, chimeric enzyme, shuffled enzyme, or combinations thereof.

### DETAILED DESCRIPTION OF THE INVENTION

Lytic enzymes and their modified forms can be used along the entire food processing chain either in place of antibiotics or to prevent the dangerous infectious bacteria from growing where antibiotics have not, or can not, be used.

The method for treating food stuffs comprises treating the food stuffs with an anti-infection agent comprising an effective amount of at least one lytic enzyme produced by a bacteria infected with a bacteriophage specific for the bacteria, a holin enzyme, chimeric enzyme, shuffled enzyme, or combinations thereof. More specifically, the lytic enzyme may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme. The lytic enzyme is preferably in an environment having a pH which allows for activity of said lytic enzyme. It is preferred that the lytic enzyme be in a carrier.

The lytic enzyme, a holin enzyme, chimeric enzyme, shuffled enzyme, or combinations thereof can be used for the treatment or prevention of various strains of Staphylococcus, Streptococcus, Listeria, Salmonella, E. coli, Campylobacter, Pseudomonas, Brucella, other bacteria, and any combination thereof. The holin enzyme, chimeric enzyme.

Antibiotics in animal feed can be readily replaced with lytic enzymes, holin enzymes, chimeric enzymes, shuffled enzymes, or combinations thereof. The lytic enzymes and their variations can be for a variety of bacteria which are found in animal feed. When applied to the feed, the lytic enzymes and their variations will kill the bacteria for which the lytic enzyme is specific. When the animal ingests the feed, there will be no adverse effects of the lytic enzyme to the animal. The protection afforded to the, feed will be transferred to the animal, except for those lytic enzymes and modified forms digested in the animal's digestive tract.

Animal feeds can be either “dry” or “wet.” It is quite common that the animal feed is in the form of a thick slurry. In those instances, prior to feeding the animals, at least one lytic enzyme, a holin enzyme, chimeric enzyme, shuffled enzyme, or combinations thereof is added and mixed into the slurry. The enzyme(s) can be lyophilized or dehydrated. However, the lytic enzyme(s) added can also be in a carrier. Alternatively, during the processing of the feed stock, the feed can be bathed in a lytic enzyme bath, prior to packaging or prior to use. The feed can also be sprayed after it is placed in the feeding pen or trough.

The carrier for the enzyme(s) may be water, an oil immersion, micelles, micelles in water or oil, liposomes, liposome in oil or water, combinations thereof, or any other convenient carrier. The enzyme(s) may be encapsulated in a carbohydrate or starch like structure, or the micelles or liposomes may be encapsulated by a starch or carbohydrate type structure. The carrier may also be in the form of a powder. The taste and texture of the carrier should be pleasing to the animal, so that the animal does not reject the food.

Prior to, or at the time the lytic enzyme(s) a holin enzyme, chimeric enzyme, shuffled enzyme, or combinations thereof is put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1. It is to be noted that some enzymes may have optimum pH's outside of this range.

The stabilizing buffer should allow for the optimum activity of the lytic enzyme, a holin enzyme, chimeric enzyme, shuffled enzyme, or combinations thereof. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

Means of application include, but are not limited to direct, indirect, carrier and special means or any combination of means.

The effective dosage rates or amounts of the lytic enzyme and its modified forms to treat bacteria will depend in part on whether the lytic enzyme, a holin enzyme, chimeric enzyme, shuffled enzyme, or combinations thereof will be used therapeutically or prophylactically, the duration of exposure of the to the infectious bacteria, the size and weight of the animal being fed, etc.

It is recognized that the antibiotic administered in the feed is used, in part, preventively, so that when an animals sticks its mouth and nose into the feed trough, it gets a high dosage of antibiotics in its mouth and nasal passages. The dosage of the lytic enzymes, a holin enzyme, chimeric enzyme, shuffled enzyme, or combinations thereof can be high enough to serve the same function. The concentration of the active units of enzyme believed to provide for an effective amount or dosage of enzyme may be in the range of about 100 units/ml to about 500,000 units/ml of fluid in the wet or damp environment of the nasal and oral passages, and possibly in the range of about 100 units/ml to about 100,000 units/ml, and more preferably in the range of about 100 units/ml to about 10,000 units/ml.

Livestock which can be fed feed which has been treated with lytic enzymes, a holin enzyme, chimeric enzyme, shuffled enzyme, or combinations thereof include, cattle, sheep, chickens, hogs, and any other livestock.

Bacterial infections of human food stuffs often occurs in the slaughterhouse, after the animal has been killed. Chickens on the processing assembly line are often dipped in a water bath, derisively referred to in the industry as “fecal soup” because the internal organs and waste of the dead chickens have fallen into this bath. Consequently, many of the chickens coming off the assembly line are contaminated prior to being packaged and shipped to market. Sometimes the chickens arrive in the grocery store, already spoiled. Other times, the consumer does not thoroughly cook the chicken, at least to a temperature to kill all bacteria present, and consequently the consumer gets food poisoning.

Lytic enzymes, a holin enzyme, chimeric enzyme, shuffled enzyme, or combinations thereof can be used to help prevent bacterial contamination of the chickens. High levels of these enzymes call be added to the water bath, thereby aiding in the killing of bacteria present. In another preferred method of preventing bacterial contamination and food poisoning, the entire chicken or parts thereof, after coming out of the water bath but prior to being packaged and shipped, can be sprayed with at least one lytic enzyme, a holin enzyme, chimeric enzyme, shuffled enzyme, or combinations thereof, to kill and prevent the growth of bacteria. It is preferred that the lytic enzyme and its modified forms for use on the chicken be specific for Salmonella or E. coli. The carrier may be water, an oil emulsion, etc. The enzyme(s) may be added in a powder. If added in powder form, it is preferred that a carrier made out of cornstarch, or some other starch be used. The powder may also be a protein powder such as a caseinate, or some other suitable substance

As before, the carrier for the lytic enzyme and its modified forms may be water, an oil immersion, micelles, reverse micelles, micelles in water or oil, liposomes, liposome in oil or water, combinations thereof, or any other convenient carrier. The lytic enzyme and its modified forms may be encapsulated in a carbohydrate or starch like structure, or the micelles or liposomes may be encapsulated by a starch or carbohydrate type structure. The carrier may also be in the form of a powder. The taste and texture of the carrier should be pleasing to the animal, so that the animal does not reject the food.

Prior to, or at the time the enzyme(s) is (are) put in the carrier system or oral delivery mode, it is preferred that the enzyme(s) be in a stabilizing buffer environment for maintaining a pH range between about 4.0 and about 9.0, more preferably between about 5.5 and about 7.5 and most preferably at about 6.1. It is to be noted that some enzymes may have optimum pH's outside of this range.

Also, as before, the stabilizing buffer should allow for the optimum activity of the lytic enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

Beef and hog carcasses are also subjected to contamination in slaughterhouses. Hence, the carcasses of hogs, beef, and other livestock may also be treated with at least one lytic enzyme, a holin enzyme, chimeric enzyme, shuffled enzyme, or combinations thereof to kill or prevent bacterial growth. The entire carcass of the animal may be dipped in a solution or liquid containing the lytic enzyme(s), a holin enzyme, chimeric enzyme, shuffled enzyme, or combinations thereof, or preferably, the carcass may be sprayed with a solution or liquid containing the enzyme. The lytic enzyme or its modified form may also be dusted onto the carcass in a powder, as described above. In a preferred embodiment of the invention, at least one lytic enzyme or its modified form for E. coli, is used. As above, it is preferred that the enzyme be in a carrier, which is buffered for the maximum activation of the lytic enzyme(s) or their modified form and to prevent denaturation of the enzyme(s).

Carcasses are not the only form of meat which suffer from contamination. Ground beef, used in hamburgers, also have a relatively high rate of contamination, compared to the rate of contamination for the rest of the food industry. Each year, a number of people die from eating hamburgers which were undercooked and contaminated, frequently with E. coli bacteria.

Consequently, at least one lytic enzyme or its modified form(s) may be incorporated into the ground meat or ground beef. The enzyme(s) may be added during the grinding of the beef, and may be added as the meat goes through the grinder, or it may be added after the meat is ground. The enzyme(s) may be in a lyophilized or dry form, whereupon the enzyme(s) becomes re-hdyrated upon contact with the “wet” ground beef. The lyophilized or dry enzymes and their modified forms may be in a powder form, such as in a carbohydrate, cornstarch or protein powder. Alternatively, the enzyme(s) may be in any of the carriers previously described, at the pH also described above.

Eggs are also subject to contamination, particularly Salmonella contamination. However, the use of lytic enzymes and their modified forms can greatly reduce the risk of Salmonella poisoning. At least one lyophilized lytic enzyme or its modified form may be applied to the shells by dipping or soaking the eggs into a lytic enzyme solution or liquid containing at least one lytic enzyme or its modified form, or by spraying a lytic enzyme solution or liquid containing a lytic enzyme (or its modified forms) onto the shells of the eggs. The lytic enzyme or its modified form(s) may be in a water or oil based solution or liquid, with the enzyme(s) either being directly in the solution or liquid, or being in a micelle, reverse micelles, liposomes, or combinations, thereof. It is preferred that the buffer solution be used prior to the enzyme(s) being put into solution or liquid. In fact, in all uses of the enzyme(s), it is always preferable that the carrier of the carrier or substance to which the enzyme(s) are to be added is first buffered. The carrier for the lytic enzyme(s) may be also be a powder. The powder, which may be a starch powder, a carbohydrate, or a protein powder, mall be sprinkled on the egg. Alternatively, the egg may be rolled in the powder.

Food contamination is often found at salad bars which routinely contain vegetables, fruits, boiled eggs, and cheeses. At salad bars, aside from air-borne contamination, it is regrettably not uncommon for customers to pick up a piece of food, examine it, and return it to the bin from whence it came, thereby contaminating the salad bar with bacteria.

To combat the bacteria, the salad of the salad bar may be sprayed or dusted with at least one lytic enzyme, a holin enzyme, chimeric enzyme, shuffled enzyme, or combinations thereof. In a preferred embodiment, the enzyme is sprayed on the salad, with the carrier for the lytic enzyme(s) being water. It is preferred that the water is buffered and that the pH is adjusted. Of course, the carrier for the enzymes can be an emulsion, an oil, or any other appropriate substance. The lytic enzyme, a holin enzyme, chimeric enzyme, shuffled enzyme, or combinations thereof can be in a micelle, a liposome, or in a reverse micelle. The enzyme(s) can also be placed in the salad dressing. Lytic enzymes for the bacteria Staphylococcus, Streptococcus, Listeria, Salmonella, E. coli, Campylobacter, Pseudomonas and any combinations thereof can be used to treat the salad bar.

Of course, the surfaces of the salad bar, as well as any other surface that comes in contact with food, can and should also be treated with at least one lytic enzyme, holin enzyme, chimeric enzyme, shuffled enzyme, or combinations thereof to destroy any bacteria present on these surfaces. The surfaces should be either sprayed with a solution or emulsion containing at least one enzyme, holin enzyme, chimeric enzyme, shuffled enzyme, or combinations thereof or the surfaces can be wiped down with a wiping material such as a clean cloths sponge, or rag which has been saturated with enzymes. The wiping material may be dipped into a buffered solution or liquid containing the enzymes. Alternatively, the wiping material may have the enzymes dehydrated or lyophilized on them, and the surface which is to be wiped is wetted. When the wiping material makes contact with the wet surface, the enzymes are re-hydrolized, and kill the bacteria on the surfaces being wiped.

Lytic enzymes, holin enzyme, chimeric enzyme, shuffled enzyme, or combinations thereof can also be used in canned and bottled goods to prevent bacterial growth or kill bacteria in these sealed goods. Prior to the sealing of the containers, at least one lytic enzyme, holin enzyme, chimeric enzyme, shuffled enzyme, or combinations thereof and preferably several enzymes is (are) added to the bottle or can. The can or bottle is then sealed. Any bacteria present will be killed by the appropriate lytic enzyme, holin enzyme, chimeric enzyme, shuffled enzyme, or combinations thereof. Some of the enzymes that may be used include the lytic enzymes and their modified version for bacteria Staphylococcus, Streptococcus, Listeria, Salmonella, E. coli, Campylobacter, Pseudomonas. The enzyme(s) may be added in almost any form, from lyophilized form, dehydrated form, in a carrier liquid, protected by micelles or in a liposome, etc. The solution or liquid in which the enzyme is added should be buffered.

It is particularly helpful to add lytic enzymes, holin enzyme, chimeric enzyme, shuffled enzyme, or combinations thereof in fruit juices, and to apple juice in particular. When the apples fall on the ground, they pick up E. coli bacteria. Regrettably, apples frequently are not washed before they are turned into cider or juice. Consequently, when the juice is drunk, usually by young children, there is a greater risk of illness. The addition of the lytic enzymes and their modified versions, and preferably the lytic enzyme specific for E. coli, prior to the sealing of the bottle, will diminish the risk of bacterial contamination and illness. The enzymes may be added to other potable liquids, preferably of the non-alcoholic nature.

As with all compositions described in this patent, the composition may, further include a bactericidal or bacteriostatic agent as a preservative.

Additionally, the agent may further comprise the enzyme lysostaphin for the treatment of any Staphylococcus aureus bacteria. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)). Lysostaphin, a gene product of Staphylococcus simulans, exerts a bacteriostatic and bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues.

Many modifications and variations of the present invention are possible in light of the above, teachings. It is, therefore, to be understood within the scope of the appended claims the invention may be protected otherwise than as specifically described.

What we claim is:

1) A method for the prevention of food poisoning, comprising:
administering to a food stock:
an effective amount of at least one enzyme selected from the group consisting of at least one lytic enzyme produced by a bacteria infected with a bacteriophage specific for said bacteria, at least one modified version of said at least one lytic enzyme, and combinations thereof wherein said modified version of said at least one lytic enzyme is selected from the group consisting of shuffled enzymes, chimeric enzymes, holin enzymes, and combinations thereof,
wherein said food stock is selected from the group consisting of live stock feed, eggs, salad bars, beef carcasses, chicken carcasses, food to be canned, and livestock feed.

2) The method of claim 1, wherein said food stock is livestock feed.

3) The method of claim 2, wherein said livestock feed is for the feeding of cattle.

4) The method of claim 2, wherein said livestock feed is for the feeding of chickens.

5) The method of claim 2, wherein said livestock feed is for the feeding of hogs.

6) The method of claim 2, wherein said livestock feed is for the feeding of sheep.

7) The method of claim 2, wherein said livestock feed is dry.

8) The method of claim 2, wherein said livestock feed is a slurry.

9) The method of claim 1, further comprising delivering said at least one enzyme in a carrier suitable for delivering said at least one said enzyme.

10) The method according to claim 1, wherein said at least one enzyme is specific for at least one strain of Pseudomonas.

11) The method according to claim 1, wherein said at least one enzyme is specific for Streptococcus pneumoniae.

12) The method according to claim 1, wherein said at least one enzyme is specific for Streptococcus fasciae

13) The method according to claim 1, wherein said at least one enzyme is specific for at least one strain of Listeria.

14) The method according to claim 1, wherein said at least one enzyme is specific for at least one strain of Salmonella.

15) The method according to claim 1, wherein said at least one enzyme is specific for at least one strain of E. coli.

16) The method according to claim 1, wherein said at least one enzyme is specific for at least one strain of Campylobacter.

17) The method according to claim 1, wherein said at least one enzyme is specific for at least one strain of Pseudomonas.

18) The method according to claim 1, wherein said at least one enzyme is specific for Streptococcus mutans.

19) The method according to claim 1, wherein said at least one enzyme is specific for Mycobacterium tuberculosis.

20) The method according to claim 1, wherein said at least one enzyme is specific for at least one strain of Streptococcus.
